(19) United States
(12) Patent Application Publication
(10) Pub. No.: US 2026/0185999 A1
(43) Pub. Date: Jul. 2, 2026

(54) METHOD FOR IDENTIFYING HIGH AFFINITY MONOCLONAL ANTIBODY HEAVY AND LIGHT CHAIN PAIRS FROM HIGH THROUGHPUT SCREENS OF B-CELL AND HYBRIDOMA LIBRARIES

(71) Applicant: Merck Sharp & Dohme LLC, Rahway, NJ (US)

(72) Inventors: Eberhard Durr, Quakertown, PA (US); Yaping Liu, Ambler, PA (US); Zhifeng Chen, Warrington, PA (US); Arthur Fridman, East Norriton, PA (US)

(73) Assignee: Merck Sharp & Dohme LLC, Rahway, NJ (US)

(21) Appl. No.: 19/328,781

(22) Filed: Sep. 15, 2025

Related U.S. Application Data
(63) Continuation of application No. 17/299,559, now abandoned.
(60) Provisional application No. 62/777,842, filed on Dec. 11, 2018.

(51) Int. Cl. G01N 33/68; C07K 16/12

(52) U.S. Cl. CPC G01N 33/6854; C07K 16/12

(57) ABSTRACT

A method is derived for identifying high affinity monoclonal antibody heavy and light chain pairs from high throughput screens of antibody producing cell libraries such as B-cell and hybridoma libraries. Specifically, the method relates to application of reversed immunocapture and high resolution tandem mass spectrometry for the identification of heavy and light chain pairs of binding antibodies obtained from high throughput screens of antibody producing cell libraries.

### CROSS REFERENCE TO RELATED APPLICATIONS

This application is a continuation of U.S. Ser. No. 17/299,559 filed Jun. 3, 2021, which is a National Stage Entry of International Patent Cooperation Treaty (PCT) Application No. PCT/US2019/064813, filed Dec. 6, 2019, and which claims benefit of U.S. Provisional Application No: U.S. 62/777,842, filed Dec. 11, 2018, all of which are herein incorporated by reference in its entirety.

### REFERENCE TO SEQUENCE LISTING SUBMITTED ELECTRONICALLY

The instant application contains a Sequence Listing which has been submitted electronically in XML format is a part of the specification and is hereby incorporated by reference in its entirety. The XML file, created on Aug. 25, 2025, is named “24677-US-CNT.xml” and is 60,409 bytes_in size.

### BACKGROUND OF THE INVENTION

### Field of the Invention

The present invention relates to a method for identifying high affinity monoclonal antibody heavy and light chain pairs from high throughput screens of B-cell and hybridoma libraries. Specifically, the present invention relates to application of reversed immunocapture and high resolution tandem mass spectrometry for the identification of heavy and light chain pairs of binding antibodies obtained from high throughput screens of B-cell or hybridoma libraries.

### Description of Related Art

Screening individual patient memory B-cells for cells that produce antibodies against a relevant disease molecule or antigen (target molecule) is a powerful tool for obtaining human monoclonal antibodies, which can then be evaluated for therapeutic effects.

The conversion of a B-cell screen “hit” to a functional antibody can be a labor and resource intensive effort. In a typical screen, memory B-cells are obtained from an individual and cultured under conditions that stimulate antibody expression in high throughput multiwell plates, which are subsequently screened with a target molecule to identify those wells of cultured memory B-cells that express antibodies that bind the target molecule, a “hit”. The amino acid sequences of the antibody heavy and light chain variable regions comprising the complementary determining regions (CDRs) from the antibodies comprising a “hit” are obtained by PCR amplification and sequencing of the mRNA from these cells. Next, the nucleotide sequences encoding the heavy and light chain variable regions from the antibodies of a “hit” are cloned into nucleic acid molecules encoding a suitable human antibody backbone (typically IgG1 or IgG2) for recombinant expression.

However, a “hit” in a memory B-cell screen frequently contains more than one antibody heavy and light chain pair and only one of the antibody heavy and light chain pairs is responsible for the “hit”. As a consequence, the amount of effort needed for identifying the antibody in the “hit” that binds the target molecule exponentially increases with the number of antibodies that are present in a “hit” because the association between heavy and light chains comprising a pair is not revealed by sequencing thereby requiring all heavy and light chain permutations to be tested for binding to the relevant molecule or antigen to identify the heavy and light chain pair responsible for the “hit”. For example, if four heavy and four light chains are identified by PCR sequencing to be in a “hit”, the expression and screening of 16 antibodies (all possible heavy and light chain combinations) is required to identify the heavy and light chain combination comprising the antibody that binds the target molecule. It is theoretically possible to minimize the occurrence of “hits” containing more than one species of antibody by seeding the high throughput format multiwell plates at a density of less than one cell/well, however, the number of high throughput multiwell format plates needed for the screen is substantially increased, which is undesirable and still does not fully eliminate “hits” that contain more than one species of antibody.

In light of the above, there is a need for a method that simplifies identification of the antibody in a “hit” that binds a target molecule from B-cells or a library of B-cells or a library of any other antibody producing cells.

### BRIEF SUMMARY OF THE INVENTION

The present invention provides a method for performing a screen of a library of antibody producing cells, for example, memory B-cells obtained from an individual (B-cell library), that facilitates identification of the antibody in a “hit” from the screen that binds a particular molecule or antigen (target molecule). The present invention further provides a method for performing a screen of hybridoma cells (hybridoma library) to identify the antibody therein that binds a particular molecule or antigen. The present invention enables identification of the heavy chain/light chain pair of an antibody that binds a particular target molecule of interest from a plurality of antibodies that do not bind the particular target molecule in a mixture comprising the antibodies. The present invention is particular useful when screening B-cell or hybridoma libraries where the cells comprising the library are plated on multiwell tissue culture plates comprising a multiplicity of wells at an average of one cell/well wherein some wells will inadvertently contain more than one cell or plated on multiwell tissue culture plates comprising a multiplicity of wells at an average of greater than one cell per well, e.g., 1.4 or more cells/well. The present invention avoids the currently practiced method for identifying the heavy chain/light chain pair of an antibody that binds a particular target molecule of interest from a plurality of antibodies that do not bind the particular target molecule when present as discussed in the description of the related art. The present invention further allows B-cell and hybridoma libraries to be plated on multiwell tissue culture plates comprising a multiplicity of wells at a cell density per well that is greater than an average of one cell per well.

The method uses reversed immunocapture (RIC) in combination with high resolution tandem mass spectrometry to directly identify the amino acid sequences of the heavy chain and light chain pair of the particular antibody species of a plurality of antibody species in a “hit” from the screen that binds the target molecule and is responsible for the “hit” in the screen.

In brief, the target molecule used to identify a “hit” in a screen of a library of B-cells or hybridoma cells is immobilized on a solid support, which allows RIC of femto-molar amounts of the intact antibody that binds the target molecule directly from plasmablast culture fluid that is available from the initial library screen. The captured antibodies are then digested using one or more proteases and the disulfide bonds reduced to provide peptide fragments of the antibodies. The peptide mass spectra of the peptides obtained by the protease digestion are then measured using nano liquid chromatography in combination with high resolution tandem mass spectrometry. The endoprotease pepsin provides sufficient sequence spectral information to cover the complementary determining regions (CDRs) of the antibody heavy chain and light chain. Mass spectrometry spectra are then compared against all antibody heavy chain and light chain sequences that had been obtained by sequencing of plasmablast mRNA to identify the heavy chain and light chain pair that provides the antibody in the “hit” that binds the target molecule.

Therefore, in one embodiment, the present invention provides a method for identifying an antibody variable heavy (V_{H}) chain and variable light (V_{L}) chain pair specific for binding an antigen of interest from a mixture of antibody producing cells, comprising the steps of (a) providing a mixture of antibody producing cells dispensed into the wells of one or more multiwell plates at a density of more than 1 cell/well wherein the antibody producing cells are grown in a culture medium under conditions sufficient for the antibody producing cells to express antibodies into the culture medium; (b) obtaining a first sample of culture medium from each of the wells and reacting the culture medium with the antigen of interest to identify those wells that contain antibodies that bind the antigen of interest; (c) determining the amino acid sequences of the V_{H} chains and V_{L} chains of the antibodies in the wells of step (b) containing antibodies that bind the antigen of interest; (d) obtaining a second sample of culture medium from those wells that contain antibodies that bind the antigen of interest and contain more than one species of V_{H} chain or V_{L} chain from step (c), reacting the culture medium with the antigen of interest immobilized to a solid support to bind and separate the antibodies that bind the antigen of interest from antibodies that do not bind the antigen of interest; (e) releasing the antibodies bound to the antigen of interest on the solid support, digesting the antibodies with one or more proteases to produce peptide fragments, and reducing disulfide bonded peptide fragments with a reducing agent to produce reduced peptide fragments; and (f) identifying the amino acid sequences of the reduced peptide fragments from step (e) with tandem mass spectrometry by comparing the tandem mass spectra for each of the reduced peptide fragments to tandem mass spectra for each of the peptides calculated to be produced from the same protease digest of step (e) for each of the V_{H} and V_{L} chains from the wells of step (d) containing antibodies that bind the antigen of interest to identify the antibody V_{H} chain and V_{L} chain pair comprising the antibody specific for binding the antigen of interest. In particular embodiments, the reducing agent is tris(2-carboxyethyl)phosphine (TCEP).

In a further embodiment, the antibody producing cells are dispensed into the wells of the one or more multiwell plates at a density of about 1.4 cells or more/well. In particular embodiments, the cells are dispensed at a density of about 2 or more cells/well. In particular embodiments, the antibody producing cells are dispensed into the wells of the one or more multiwell plates at a density of from about 1.4 cells/well to about 10 cells/well.

In particular embodiments, the one or more proteases is pepsin.

In particular embodiments, determining the amino acid sequences of the V_{H} chains and V_{L} chains of the antibodies in the wells of step (c) containing antibodies that bind the antigen of interest comprises obtaining nucleic acid molecules from the antibody producing cells of the wells containing the antibodies that bind the antigen of interest, sequencing the nucleic acid molecules encoding the antibodies to provide polynucleotide sequences encoding the V_{H} and V_{L} chains of the antibodies, and translating the polynucleotide sequences encoding the V_{H} and V_{L} chains of the antibodies to determine the amino acid sequences of the V_{H} chains and V_{L} chains of the antibodies.

In particular embodiments, the nucleic acid molecules are obtained from the antibody producing cells in step (c) by reverse transcription polymerase chain reaction (RT-PCR) amplification of cDNA encoding the antibodies. In further embodiments, the RT-PCR is performed with a primer pair comprising an upstream PCR primer and a downstream PCR primer wherein the melting temperature difference (ΔTm) between the upstream primer and the downstream primer is about 3° C. or less. In further embodiments, each well is assigned a specific nucleotide sequence identifier and one or both of the upstream and downstream PCR primers for each well comprises the specific nucleotide sequence identifier assigned to the well.

In particular embodiments of the present invention, the antigen of interest is covalently linked to biotin and the antigen of interest covalently linked to the biotin is immobilized to a solid support coated with streptavidin. In particular embodiments, the solid support comprises magnetic beads, which are coated with the streptavidin.

In particular embodiments of the present invention, the antibody producing cells comprise memory B-cells or hybridoma cells. In particular embodiments, the antibody library comprises nucleic acid molecules encoding human or animal antibody VH and VL chains. In particular embodiments, the antibody producing cells produce human antibodies or animal antibodies. In particular embodiments, the animal antibodies are camelid antibodies, avian antibodies, rabbit antibodies, murine antibodies, equine antibodies, rat antibodies, bovine antibodies, feline antibodies, canine antibodies, or shark antibodies.

The present invention further provides a method for determining the amino acid sequences of an antibody V_{H} chain and V_{L} chain pair specific for binding an antigen of interest from other antibodies produced by a mixture of antibody producing cells, comprising (a) providing a cell culture of a mixture of antibody producing cells derived from two or more starting antibody producing cells, wherein each starting antibody producing cell expresses an antibody and wherein one of the two or more starting antibody producing cells expresses the antibody specific for the antigen of interest and the other two or more starting antibody producing cells express antibodies that do not bind the antigen of interest; (b) determining the amino acid sequences of the V_{H} and V_{L} chains for each of the antibodies expressed by the antibody producing cells in the cell culture of step (a); (c) obtaining the antibodies that bind the antigen of interest from the cell culture of step (a) by contacting the cell culture with the antigen of interest immobilized to a solid support to separate the antibodies that bind the antigen of interest from those antibodies that do not bind the antigen of interest; (d) digesting the antibodies that bind the antigen of interest with one or more proteases to produce peptide fragments and reducing disulfide bonded peptide fragments with a reducing agent to produce reduced peptide fragments; and (e) identifying the amino acid sequences of the reduced peptide fragments from step (d) with tandem mass spectrometry by comparing the tandem mass spectra for each of the reduced peptide fragments to tandem mass spectra for each of the peptides calculated to be produced from the same protease digest of step (d) for each of the V_{H} and V_{L} chains from step (c) to identify the antibody V_{H} chain and V_{L} chain pair comprising the antibody specific for binding the antigen of interest. In particular embodiments, the reducing agent is tris(2-carboxyethyl)phosphine (TCEP). In particular embodiments, the one or more proteases is pepsin. In particular embodiments, the mixture of antibody producing cells is derived from two to 10 starting cells.

In particular embodiments, determining the amino acid sequences of the V_{H} chains and V_{L} chains of the antibodies of step (b) comprises obtaining nucleic acid molecules from the antibody producing cells, sequencing the nucleic acid molecules encoding the antibodies to provide polynucleotide sequences encoding the V_{H} and V_{L} chains of the antibodies, and translating the polynucleotide sequences encoding the V_{H} and V_{L} chains of the antibodies to determine the amino acid sequences of the V_{H} chains and V_{L} chains of the antibodies.

In particular embodiments, the nucleic acid molecules are obtained from the antibody producing cells in the cell culture by RT-PCR amplification of cDNA encoding the antibodies. In further embodiments, the RT-PCR is performed with a primer pair comprising an upstream PCR primer and a downstream PCR primer wherein the melting temperature difference (ΔTm) between the upstream primer and the downstream primer is about 3° C. or less. In further embodiments, the cell culture is assigned a specific nucleotide sequence identifier and one or both of the upstream and downstream PCR primers for cell culture comprises the specific nucleotide sequence identifier assigned to the well.

In particular embodiments of the present invention, the antigen of interest is covalently linked to biotin and the antigen of interest covalently linked to the biotin is immobilized to a solid support coated with streptavidin. In particular embodiments, the solid support comprises magnetic beads, which are coated with the streptavidin.

In particular embodiments of the present invention, the antibody producing cells comprise memory B-cells or hybridoma cells. In particular embodiments, the antibody library comprises nucleic acid molecules encoding human or animal antibody VH and VL chains. In particular embodiments, the antibody producing cells produce human antibodies or animal antibodies. In particular embodiments, the animal antibodies are camelid antibodies, avian antibodies, rabbit antibodies, murine antibodies, equine antibodies, rat antibodies, bovine antibodies, feline antibodies, canine antibodies, or shark antibodies.

The present invention further provides a method for obtaining the amino acid sequences of an antibody or fragment thereof specific for an antigen of interest from the amino acid sequences of other antibodies produced by a mixture of antibody producing cells, comprising (a) providing a cell culture of a mixture of antibody producing cells derived from two or more starting antibody producing cells, wherein each starting antibody producing cell expresses an antibody and wherein one of the starting antibody producing cells expresses the antibody specific for the antigen of interest and the other starting antibody producing cells express antibodies that do not bind the antigen of interest; (b) determining the amino acid sequences of each of the antibodies expressed by the antibody producing cells in the cell culture; (c) obtaining the antibodies that bind the antigen of interest from the cell culture by reacting the cell culture with the antigen of interest immobilized to a solid support to separate the antibodies that bind the antigen of interest from the antibodies that do not bind the antigen of interest; (d) digesting the antibodies that bind the antigen of interest with one or more proteases to produce peptide fragments and reducing disulfide bonded peptide fragments with a reducing agent to produce reduced peptide fragments; and (e) identifying the amino acid sequences of the reduced peptide fragments from step (d) with tandem mass spectrometry by comparing the tandem mass spectra for each of the reduced peptide fragments to tandem mass spectra for each of the peptides calculated to be produced from the same protease digest of step (d) for each of the V_{H} and V_{L} chains from step (b) to determine the amino acid sequences of the antibody or fragment thereof specific for the antigen from the mixture of antibodies. In particular embodiments, the reducing agent is tris(2-carboxyethyl)phosphine (TCEP). In particular embodiments, the one or more proteases is pepsin. In particular embodiments, the mixture of antibody producing cells is derived from two to 10 starting cells.

In particular embodiments, determining the amino acid sequences of the antibodies in step (b) comprises obtaining nucleic acid molecules from the antibody producing cells, sequencing the nucleic acid molecules encoding the antibodies to provide polynucleotide sequences encoding the antibodies, and translating the polynucleotide sequences encoding the antibodies to determine the amino acid sequences of the antibodies.

In particular embodiments, the nucleic acid molecules are obtained from the antibody producing cells in the cell culture by RT-PCR amplification of cDNA encoding the antibodies. In further embodiments, the RT-PCR is performed with a primer pair comprising an upstream PCR primer and a downstream PCR primer wherein the melting temperature difference (ΔTm) between the upstream primer and the downstream primer is about 3° C. or less. In further embodiments, the cell culture is assigned a specific nucleotide sequence identifier and one or both of the upstream and downstream PCR primers for cell culture comprises the specific nucleotide sequence identifier assigned to the well.

In particular embodiments of the present invention, the antigen of interest is covalently linked to biotin and the antigen of interest covalently linked to the biotin is immobilized to a solid support coated with streptavidin. In particular embodiments, the solid support comprises magnetic beads, which are coated with the streptavidin.

In particular embodiments of the present invention, the antibody producing cells comprise memory B-cells or hybridoma cells. In particular embodiments, the antibody library comprises nucleic acid molecules encoding human or animal antibodies. In particular embodiments, the antibody producing cells produce human antibodies or animal antibodies. In particular embodiments, the animal antibodies are camelid antibodies, avian antibodies, rabbit antibodies, murine antibodies, equine antibodies, rat antibodies, bovine antibodies, feline antibodies, canine antibodies, or shark antibodies.

### BRIEF DESCRIPTION OF THE DRAWINGS

FIG. 1A shows the molecular structure of β-1, 6-linked Poly-N-acetyl-D-glucosamine. Some of the N-acetyl groups of the natural exopolysaccharide are deacetylated.

FIG. 1B shows the molecular structure of synthetic PNAG five-mer with linker and biotin anchor.

FIG. 1C shows the molecular structure of synthetic dPNAG five-mer with linker and biotin anchor.

FIG. 2 shows a schematic work flow of the B-cell screen in conjunction with the rIC-MS method for antibody heavy chain and light chain identification.

FIG. 3 shows a schematic workflow of the reversed immunocapture (rIC) in combination with high resolution tandem mass spectrometry (1D nanoLC-MS/MS.

FIG. 4 shows an example of the mass spectrometry data for peptide Z001-VL5 having the amino acid sequence ISCSGSSSNIGSN (SEQ ID NO: 16).

FIG. 5 shows ELISA results for each of the nine heavy chain and light chain pairs possible from the VH domain and VL domain amino acid sequences identified by NGS of the mRNA cell lysates made from the cells in the PNAG positive well C001.

FIG. 6 shows ELISA results for each of the six heavy chain and light chain pairs possible from the VH domain and VL domain amino acid sequences identified by NGS of the mRNA cell lysates made from the cells in the PNAG positive well Z001.

### DETAILED DESCRIPTION OF THE INVENTION

### Definitions

As used herein, “antibody” refers to an entire immunoglobulin, including recombinantly produced forms and includes any form of antibody that exhibits the desired biological activity. Thus, it is used in the broadest sense and specifically covers, but is not limited to, monoclonal antibodies (including full length monoclonal antibodies) obtained from B-cells of human or animal origin, including but not limited to llama, camels, chickens, rabbits, mice, rats, horses, sharks, cows, and alpacas.

An “antibody” is a glycoprotein comprising at least two heavy chains (HCs) and two light chains (LCs) inter-connected by disulfide bonds, or an antigen binding portion thereof. Each heavy chain is comprised of a heavy chain variable region or domain (abbreviated herein as V_{H}) and a heavy chain constant region or domain. In certain naturally occurring IgG, IgD and IgA antibodies, the heavy chain constant region is comprised of three domains, C_{H}1, C_{H}2 and C_{H}3. In certain naturally occurring antibodies, each light chain is comprised of a light chain variable region or domain (abbreviated herein as V_{L}) and a light chain constant region or domain. The light chain constant region is comprised of one domain, CL. The human V_{H} includes six family members: V_{H}1, V_{H}2, V_{H}3, V_{H}4, V_{H}5, and V_{H}6 and the human V_{L} family includes 16 family members: V_{κ}1, V_{κ}2, V_{κ}3, V_{κ}4, V_{κ}5, V_{κ}6, V_{λ}1, V_{λ}2, V_{λ}3, V_{λ}4, V_{λ}5, V_{λ}6, V_{λ}7, V_{λ}8, V_{λ}9, and V_{λ}10. Each of these family members can be further divided into particular subtypes.

The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDR regions and four FR regions, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. The assignment of amino acids to each domain is, generally, in accordance with the definitions of Sequences of Proteins of Immunological Interest, Kabat, et al.; National Institutes of Health, Bethesda, Md.; 5^{th} ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

In general, while an antibody comprises six CDRs, three on the V_{H} and three on the V_{L}, the state of the art recognizes that in most cases, the CDR3 region of the heavy chain is the primary determinant of antibody specificity, and examples of specific antibody generation based on CDR3 of the heavy chain alone are known in the art (e.g., Beiboer et al., J. Mol. Biol. 296: 833-849 (2000); Klimka et al., British J. Cancer 83: 252-260 (2000); Rader et al., Proc. Natl. Acad. Sci. USA 95: 8910-8915 (1998); Xu et al., Immunity 13: 37-45 (2000). See Kabat et al. (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196: 901-917 (defining the CDR regions of an antibody by structure).

The following general rules shown in Table 1 may be used to identify the CDRs in an antibody sequence. There are rare examples where these virtually constant features do not occur; however, the Cys residues are the most conserved feature.

TABLE 1

| | |
|---|---|
| Light chain | |
| CDR1 | |
| Start | About amino acid residue 24 |
| Residue before | Usually a Cys |
| Residue after | Usually a Trp. Typically Trp-Tyr-Gln, but also, |
| | Trp-Leu-Gln, Trp-Phe-Gln, or Trp-Tyr-Leu |
| Length | 10 to 17 amino acid residues |
| Light chain | |
| CDR2 | |
| Start | Usually 16 amino acid residues after the end of CDR1 |
| Residues before | Generally Ile-Tyr, but also, Val-Tyr, Ile-Lys, or |
| | Ile-Phe |
| Length | Usually seven amino acid residues |
| Light chain | |
| CDR3 | |
| Start | Usually 33 amino acid residues after end of CDR2 |
| Residue before | Usually Cys |
| Residues after | Usually Phe-Gly-Xaa-Gly (SEQ ID NO: 221) |
| Length | Seven to 11 amino acid residues |
| Heavy chain | |
| CDR1 | |
| Start | About amino acid residue 26 (usually four amino acid |
| | residues after a Cys) [Chothia/AbM definition]; |
| | Kabat definition starts five amino acid residues later |
| Residues before | Usually Cys-Xaa-Xaa-Xaa (SEQ ID NO: 222) |
| Residues after | Usually a Trp. Typically Trp-Val, but also, Trp-Ile |
| | or Trp-Ala |
| Length | 10 to 12 amino acid residues [AbM definition]; |
| | Chothia definition excludes the last four amino acid |
| | residues |
| Heavy chain | |
| CDR2 | |
| Start | Usually 15 amino acid residues after the end of |
| | Kabat/AbM definition) of heavy chain CDR1 |
| Residues before | Typically Leu-Glu-Trp-Ile-Gly (SEQ ID NO: 223), but |
| | a number of variations |
| Residues after | Lys/Arg-Leu/Ile/Val/Phe/Thr/Ala-Thr/Ser/Ile/Ala |
| Length | Kabat definition 16 to 19 amino acid residues; AbM |
| | (and recent Chothia) definition ends seven amino acid |
| | residues earlier |
| Heavy chain | |
| CDR3 | |
| Start | Usually 33 amino acid residues after end of heavy |
| | chain CDR2 (usually two amino acid residues after |
| | a Cys) |
| Residues before | Usually Cys-Xaa-Xaa (typically Cys-Ala-Arg) |
| Residues after | Usually Trp-Gly-Xaa-Gly (SEQ ID NO: 224) |
| Length | Three to 25 amino acid residues |

In general, the basic antibody structural unit comprises a tetramer. Each tetramer includes two identical pairs of polypeptide chains, each pair having one “light” chain (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function of the antibody. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology, Ch. 7 (Paul, W., ed., 2nd ed. Raven Press, N.Y. (1989).

As used herein, “B-cells” also known as “B lymphocytes”, area a type of white blood cell of the lymphocyte subtype. They function in the humoral immunity component of the adaptive immune system by secreting antibodies. Additionally, B cells present antigen (they are also classified as professional antigen-presenting cells (APCs)) and secrete cytokines. In mammals, B cells mature in the bone marrow, which is at the core of most bones. In birds, B cells mature in the bursa of Fabricius, a lymphoid organ. B-cell types include the following:

Plasmablasts—short-lived, proliferating antibody-secreting cell arising from B cell differentiation. Plasmablasts are generated early in an infection and their antibodies tend to have a weaker affinity towards their target antigen compared to plasma cells. Plasmablasts can result from T cell-independent activation of B cells or the extrafollicular response from T cell-dependent activation of B cells.

Plasma cells—long-lived, non-proliferating antibody-secreting cell arising from B cell differentiation. There is evidence that B cells first differentiate into a plasmablast-like cell, then differentiate into a plasma cell. Plasma cells are generated later in an infection and, compared to plasmablasts, have antibodies with a higher affinity towards their target antigen due to affinity maturation in the germinal center (GC) and produce more antibodies. Plasma cells typically result from the germinal center reaction from T cell-dependent activation of B cells, however they can also result from T cell-independent activation of B cells.

Memory B cells—Dormant B cells arising from B cell differentiation. Their function is to circulate through the body and initiate a stronger, more rapid antibody response (known as the anamnestic secondary antibody response) if they detect the antigen that had activated their parent B cell (memory B cells and their parent B cells share the same B-cell receptors, thus they detect the same antigen). Memory B cells can be generated from T cell-dependent activation through both the extrafollicular response and the germinal center reaction as well as from T cell-independent activation of B1 cells.

As used herein, “B-cell library” or “library of B-cells” refers to a collection or mixture of B-cells that have been obtained from an individual.

As used herein, “library of antibody producing cells” or “antibody producing cell library” refers to a collection or mixture of antibody producing cells.

As used herein, the term “antibody producing cell” refers to a cell that produces an antibody. Examples of antibody producing cells include but are not limited to B-cells and hybridoma cells.

As used herein, “specifically binds” refers, with respect to an antigen or molecule, to the preferential association of an antibody or other ligand, in whole or part, with the antigen or molecule and not to other molecules, particularly molecules found in human blood or serum. Antibodies typically bind specifically to their cognate antigen with high affinity, reflected by a dissociation constant (K_{D}) of 10⁻⁷ to 10⁻¹¹ M or less. Any K_{D} greater than about 10⁻⁶ M is generally considered to indicate nonspecific binding.

The term “Next-Generation Sequencing” or “NGS” or “high-throughput sequencing” or “HTS”, refers to a number of different modern sequencing technologies including but not limited to Illumina (Solexa) sequencing, Roche 454 sequencing, Ion torrent, Proton/PGM sequencing, and SOLiD sequencing.

The term “high resolution tandem mass spectrometry” or (“M/M” or “M²”) refers to a process wherein in a tandem mass spectrometer, ions are formed in the ion source and separated by mass-to-charge ratio in the first stage of mass spectrometry (MS1). Ions of a particular mass-to-charge ratio (precursor ions) are selected and fragment ions (product ions) are created by collision-induced dissociation, ion-molecule reaction, photodissociation, or other process.

There are several types of tandem mass spectrometers, including but not limited to triple stage quadrupoles (TSQ), 3D and linear ion traps, quadrupole/time-of-flight (QTOF) hybrid instruments, quadrupole-linear ion trap hybrid instruments (QTRAP), and time-of-flight-time-of-flight (TOF/TOF) instruments. With 3D or linear quadrupole ion traps, tandem MS can also be performed in a single mass analyzer over time and in these instruments this process may be iterated more than once to yield MSⁿ spectra. These instruments achieve fragmentation by resonance excitation, which induces collisions with the trap bath gas (helium) of sufficiently high energy to induce fragmentation. The other instruments mentioned above employ collision induced disassociation (CID) in a collision cell. Other methods that can be used to fragment molecules for tandem MS include electron capture dissociation (ECD), electron transfer dissociation (ETD), infrared multiphoton dissociation (IRMPD) and blackbody infrared radiative dissociation (BIRD). M/M may be used for the de novo sequencing of peptides or to identify peptide sequences by correlating tandem mass spectra to feasible amino acid sequences.

### EMBODIMENTS

Antibodies are amongst the fastest growing classes of biomolecules in the in the therapeutic industry (Ecker et al., mAbs 7: 9-14 (2015)). High throughput screening of the B-cell repertoire of disease afflicted or convalescent patients is a powerful tool to directly obtain individual antibody secreting cells that produce immunoglobulins with high affinity to antigens of a specific pathogen (Xia et al., Oncotarget 8: 73654-73669 (2017)). Ideally, single memory B-cells are cultured in high throughput format multiwell plates and antibody binding to the desired antigen is then tested using the supernatant obtained from the short term culture. For positive wells, the corresponding antigen producing cells are then lysed, the RNA is extracted and reverse transcription polymerase chain reaction (RT-PCR) is performed to obtain either the nucleotide sequence of the entire immunoglobulin heavy and light chains or as a minimum the nucleotide sequence of the heavy and light chain variable domains comprising the complementarily determining regions (CDRs). The identified heavy and light chain pairs are then co-expressed in suitable production cell lines and the recombinant antibodies can then be further evaluated.

In order to keep the number of high throughput multiwell plates within a manageable quantity, the actual average cell seeding density typically exceeds one antigen secreting cell per well. However, this has downstream complications for the pairing of heavy and light chain sequences because the RT-PCR reaction may now identify multiple heavy and light chain sequences in a well that screens positive for a target antigen, for example, such a well may contain up to four (or theoretically even more) heavy and/or light chain sequences representing, in the case of four heavy chains and four light chains, 16 potential antibody species. Because the heavy and light chain sequences originate from independent genes, when there is more than one antibody species in the well, there is no pairing information that can be obtained from the RT-PCR experiment that would enable identification of the particular antibody species among the other potential antibody species that is responsible for binding the target antigen. Thus, to identify the correct heavy and light chain pair for the antibody species that binds to the target antigen from all the other potential heavy and light chain pairs, each possible heavy and light chain combination has to be cloned into a host cell, expressed from the host cell, and tested for binding to the target antigen, which can entail a considerable expenditure of time and resources.

Cheung et al., Nat. Biotechnol. 30:447-452 (2012) describes a method that relies upon tandem mass spectrometry to identify peptides for all antibodies from a blood sample from an individual that bind an antigen of interest, the method still does not provide a method for identifying the correct heavy chain and light chain pair without generating every potential heavy and light chair and screening each pair for binding to the antigen. Boutz et al., Analyt. Chem. 86: 4758-4766 (2014) discloses a shotgun proteomics approach to antibody identification relies on the integration of two main experimental pipelines: (1) high throughput sequencing of B lymphocyte cDNAs to generate a database of class-switched antibody variable domain sequences in a particular individual; (2) a protein biochemistry and mass spectrometry-based proteomics pipeline for the identification of peptides derived from antigen-specific antibodies. However, neither approach demonstrated a method that could be used for identification of a particular antibody binding an antigen of interest in a sample from other antibodies in the sample.

To solve this problem, the inventors have discovered a method that simplifies the process and is an improvement to the screen of B-cells and B-cell libraries obtained from an individual since it enables the ability to quickly identify antibodies that bind a target antigen from all other non-binding antibodies when present. The method further enables B-cell screens to be conducted at higher plating densities than otherwise would be used. Thus, B-cell screens may be performed under conditions where the wells of the multiwell plates are seeded at a cell density of more than 1 cell/well, or about 1.4 cells/well to about 2 cells/well, or about 1.4 cells/well to about 5 cells/well. In particular embodiments the multiwell plates are seeded at a density of at least 1.4 cells/well. The inventors have further discovered that method simplifies the process of screening hybridoma cells or hybridoma libraries for the hybridoma cell that specifically produces the antibody of interest.

The improvement provided by the present invention is the use of reversed immunocapture in combination with high resolution tandem mass spectrometry (rIC-MS/MS) to directly identify the heavy and light chain pair of the antibody that binds a target antigen from all other non-target binding antibodies in the B-cell or hybridoma cell culture supernatant fraction from B-cell or hybridoma screens that are positive for binding the target antigen. The addition of the rIC-MS/MS step to the B-cell or hybridoma cell screening step provides a rapid, sensitive method for identifying antibodies by sequence that are specific for a particular target antigen. While the method is exemplified herein using a bacterial exopolysaccharide as the target antigen, the method works for all classes of biological antigens such as proteins and lipids and works regardless as whether the epitope the antibody recognizes is linear or conformational.

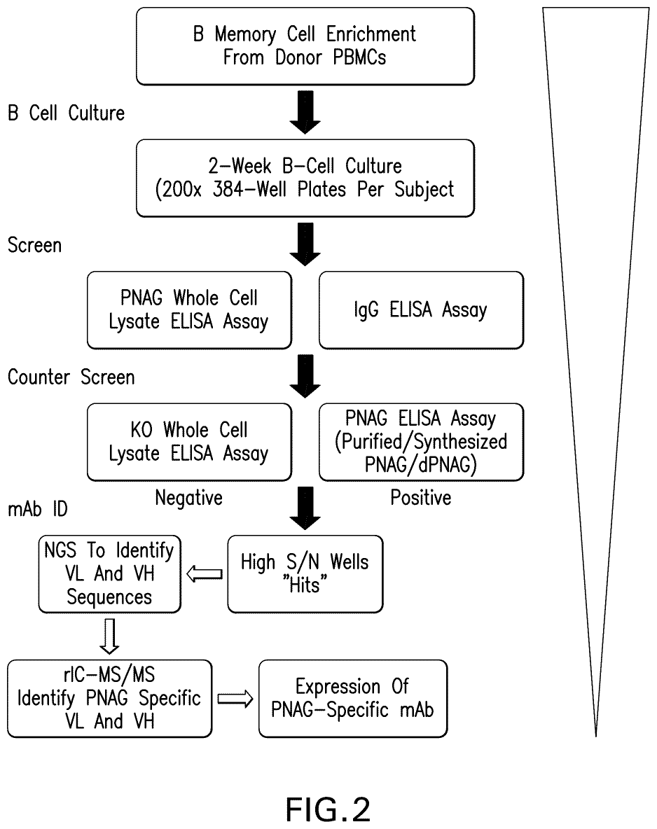

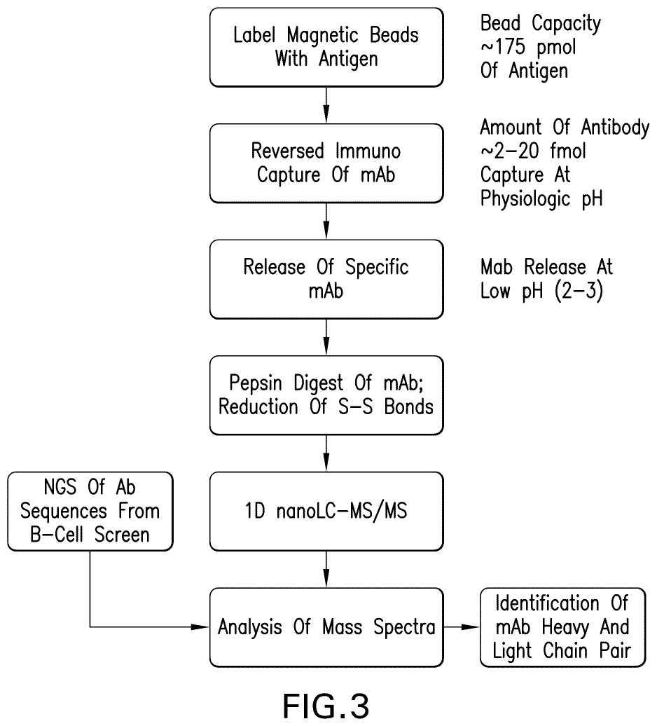

A flow diagram showing the two steps comprising the operation of the present invention is shown in FIG. 2 and FIG. 3 with respect to screening B-cells. A screen of hybridoma cells would follow a similar scheme. A first step as shown in FIG. 2 comprises culturing memory B-cells from donor peripheral blood mononuclear cells (PBMCs) from an individual under conditions that are suitable for enriching memory B-cells. For example, memory B-cells may be seeded into the wells of multiwell plates at an average of 1.4 or more B-cells per well in the presence of gamma irradiated HEK293 cells expressing human CD40 ligand at about 10,000 cells per well and recombinant human IL-21. In a typical B-cell screen, B-cells are cultivated for about two weeks; at about 200 384-well multiwell plates per individual.

For the screen, the cell culture fluid from each of the wells is tested in an enzyme linked immunosorbent assay (ELISA) for binding to a target antigen. For example, in particular embodiments, the target antigen is immobilized to a multiplicity of solid supports (e.g., wells of a multiwell plate) and then incubated with the cell culture fluid for a time sufficient for any antibodies in the cell culture fluid specific for the target antigen to bind the target antigen. Thereafter, the culture fluid is removed and the immobilized target antigen is incubated with an antibody that binds IgG labeled with a detectable moiety. A control ELISA may be performed using the anti-IgG antibodies labeled with a detectable moiety. Primary hits may then be counter-screened with an appropriate negative control and rescreened with the target antigen to identify wells positive for antibodies that bind the target antigens. The B-cells are harvested from the positive wells, lysed, and the mRNA reverse-transcribed into cDNA using reverse-transcription polymerase chain reaction (RT-PCR) and the amplified cDNA subjected to next generation sequencing (NGS) to identify the nucleotide sequences encoding the variable heavy and light chains.

In a second step, as shown in FIG. 3, reversed immunocapture is used to isolate antibodies from the positive wells, which are then digested with one or more proteases. The disulfide bonds are reduced and the reduced peptide fragments subjected to high resolution tandem mass spectrometry to produce acquired mass spectrum for each of the peptides. Theoretical spectra of peptide fragments predicted to be obtained from the one or more protease digestion of the amino acid sequences obtained from NGS are prepared. Then the theoretical spectra are compared or correlated to the acquired spectra and the probability of association is calculated. A high probability of association indicates the acquired and theoretical spectra are correlated and indicates that the amino acid sequences underlying the theoretical spectra are of the antibody species that binds the target antigen. These analyses may be performed by a computer algorithm capable of creating theoretical spectra from predicted peptide sequences and comparing or correlating theoretical spectra to acquired spectra. For example, SEQUEST is a tandem mass spectrometry data analysis program used for protein identification disclosed in U.S. Pat. Nos. 6,017,693 and 5,538,897. SEQUEST identifies collections of tandem mass spectra to peptide sequences that have been generated from databases of protein sequences.

The following examples are intended to promote a further understanding of the present invention.

### Example 1

This example exemplifies use of the method of the present invention to identify human antibodies that specifically bind the bacterial antigen exopolysaccharide β-1,6-linked Poly-N-acetyl-D-glucosamine from a library of memory B-cells obtained from a human individual.

### Experimental Details and Results

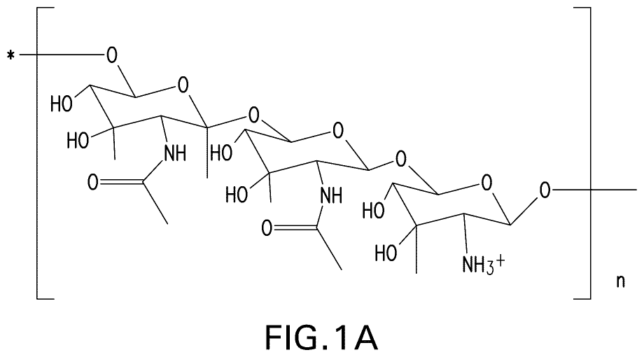

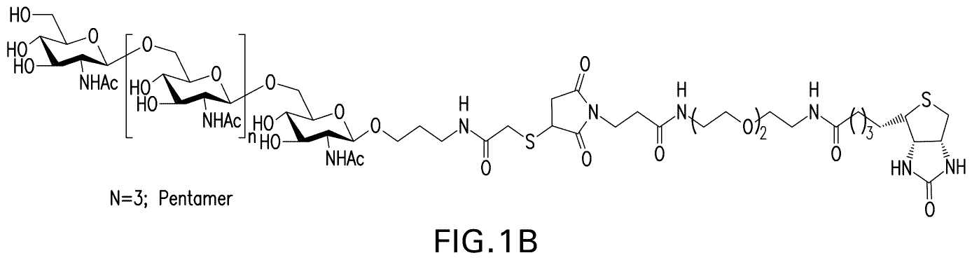

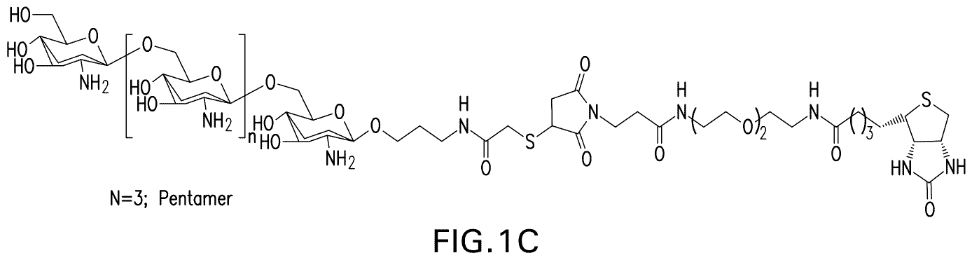

A high throughput B cell screen against the bacterial exopolysaccharide β-1, 6-linked Poly-N-acetyl-D-glucosamine (PNAG) (FIG. 1) was performed to obtain the sequences of PNAG specific human monoclonal antibodies. The B cells were obtained from a healthy human donor and the antibody secreting cells were seeded at a seeding density of 1.4 cells/well with a total of 76,800 wells. The first screen of the B-cell supernatant containing secreted antibody was performed against native PNAG that had been obtained from bacterial extracts. This initial screen resulted in the identification of 31 PNAG positive wells. A negative counter screen was performed against PNAG negative cell wall preparations, which was followed by a second positive counter screen of the B cell culture supernatants to determine their specificity towards either the unmodified acetylated form of the polysaccharide (PNAG) or its deacetylated form (dPNAG).

A work-flow of the B-cell screen is shown in FIG. 2. The oligosaccharides for the second screen were obtained by chemical synthesis and consisted of PNAG 5-mers (FIG. 1A, FIG. 1B, and FIG. 1C). Out of the 31 positive wells five wells were chosen to demonstrate the ability of the rIC/MS methodology to directly identify heavy and light chain pairs of the binding antibody out of the supernatant of the antibody secreting cells. The antibodies in two of the positive wells (C001 and Z001) preferably recognized the unmodified acetylated form of PNAG. The antibodies obtained from two different positive wells (E001 and G001) preferably recognized dPNAG. Lastly, the antibodies from one well (B001) recognized both, PNAG as well as dPNAG.

Heavy and light chain next generation sequencing (NGS) reactions performed using the cell lysates of the corresponding B cell wells of the antibody secreting cells identified a total of 12 heavy and 13 light chain sequences for the five PNAG positive wells combined (Table2).

TABLE 2

| | | | Number of | | | Sum Pep Score | | Sum Pep Score | |
|---|---|---|---|---|---|---|---|---|---|
| | | | Heavy | Light | | heavy chain | | light chain | |
| | Positive | | chains | chains | | | Fold | | Fold |
| | Well | | determined | | Number of | | better than | | better than |
| | Name | Antigen | by NGS | | Combinations | 1^{st} ID | 2^{nd} ID | 1^{st} ID | 2^{nd} ID |
| 1 | C001 | PNAG | 3 | 3 | 9 | 2.65 | ∞ | 4/79 | ∞ |
| 2 | Z001 | PNAG | 2 | 3 | 6 | 9.74 | ∞ | 17.47 | ∞ |
| 3 | B001 | PNAG/dPNAG | 1 | 1 | 1 | 0 | ∞ | 0 | ∞ |
| 4 | E001 | dPNAG | 3 | 4 | 12 | 71.50 | ∞ | 87.09 | ∞ |
| 5 | G001 | dPNAG | 3 | 3 | 9 | 23.35 | ∞ | 19.12 | ∞ |

“∞” represents the absence of a second best identification

The table shows the number of heavy and light chain sequences obtained for each positive well by NGS. The “Sum Peptide Score” obtained after rIC/MS analysis is shown for each identified antibody heavy and light chain (1^{st} ID) and compared to the second best identification (2^{nd} ID).

The amino acid sequences for the heavy chain variable (V_{H}) domains and light chain variable (V_{L}) domains identified for PNAG positive wells C001 and Z001 are shown in Table 3 and Table 4, respectively

TABLE 3
V_{H} and V_{L} domains identified for PNAG positive wells C001

| Variable | | | SEQ ID |
|---|---|---|---|
| Domain | Name | Amino Acid Sequence | NO: |
| Heavy | C001- | EVQLVESGGGLVQPGGSLRLSCAASGFTVSGSHMHWVR | 1 |
| Chain | H1 | QASGKGLEWVGHIRNIADGSATAYSASVKGRFTISRDDST | |
| | | NTAYLEMNSLRTEDTAVYYCSRQTLSCHDYWGQGALVT | |
| | | VSSASTK | |
| | C001- | QVQLQESGPRRVKPSETLSLTCSVSGGSVRSGNYYWNWI | 2 |
| | H2 | RQPPGKGLEWIGYIFDSGTTDYNPSLKSRVTMSVDTSKN | |
| | | QFSLQLRSVTTADTAVYYCVREEGKLGDCSDGSCRRKYF | |
| | | WFDPWGQGTLVTVSSASTK | |
| | C001- | EVQLVQSGGEVKKPGESLRISCKGSGYSFTNYWISWVRQ | 3 |
| | H3 | MPGKGLEWMGKIDPSDSYTKYSPSFQGHVTISADKSISTA | |
| | | YLQWSSLKASDTAMYYCARHGSRDVVEVPAAFNWFDP | |
| | | WGQGTLVTVSSASTK | |
| Light | C001- | EIVLTQSPGTLSLSPGERATLSCRASQSVYGSYLAWYQQK | 4 |
| Chain | K1 | PGQAPRLLIYHASIRATGIPDRFSGSGSGTDFTLTISRLEPE | |
| | | DFAVYYCQQYGSSPPYTFGPGTKVDIKR | |
| | C001- | DIQMTQSPSSLSASLGDRVIITCRASQNINRNLNWYQQKP | 5 |
| | K2 | GKAPQLLIYAASTLQSGVPSRFSGSGSGTDFSLTISSLQPG | |
| | | DFATYYCQQSSSTPRTFGQGTKVEIKRTIGKV | |
| | C001- | QSALTQPRAVSGSPGQSVTISCTGTSSNVGRYNYVSWYQ | 6 |
| | L1 | QRPGKAPTLMIYDVSERPSGVPDRFSGSKSGNTASLTISGL | |
| | | QADDEADYYCCSYAGSYTLLFGGGTKLTVLGQPK | |

H = heavy chain variable region, K = kappa variable domain, and L = lambda variable domain

TABLE 4
V_{H} and V_{L} domains identified for PNAG positive well Z001

| Variable | | | SEQ ID |
|---|---|---|---|
| Domain | Name | Amino Acid Sequence | NO: |
| Heavy | Z001- | EVQLVESGGGLVQPGGSLRLSCAASGFSFTYAMSWVRQA | 7 |
| chain | H1 | PGKGLEWVSAISETGDYIYYADSVKGRFTISRDYSKSTLYL | |
| | | QMNSLRAGDTAVYYCAKDSCSHYDCYTRGFGFDIWGQG | |
| | | TMVTVSSASTK | |
| | Z001 | QVQLVQSGAEVRKPGSSMKVSCKASGGTFSTYGFHWVRE | 8 |
| | H2 | APGQGLEWVGGIMVVFDTTYYAQKFQGRVTITADEFTST | |
| | | VYMEMSSLTSEDTAVYYCARDPYYDYEQGQWDVWGKG | |
| | | TTVTVSSASTK | |
| Light | Z001- | DIQMTQSPSSVSASVGDRVTITCRASQGISSWLAWYQQKP | 9 |
| chain | K1 | GKAPKLLIYAASSLQSGVPSRFSGSGSGTDFTLTISSLQPED | |
| | | FATYYCQQANSFPVTFGQGTRLEIKR | |
| | Z001- | QSVLTQPPSASGTPGQTVTISCSGSSSNIGSNTVNWYQHLP | 10 |
| | L1 | GTAPKLLIHNNDQRPSGVPDRFSASKSGTSASLAISGLQSD | |
| | | DEADYYCAVWDDSLEGGLFGGGTKLTVLGQPK | |
| | Z001- | QAVLTQPSSLSASPGASASLTCTLHSGINVATYRIFWYQQK | 11 |
| | L2 | PGSPPHYLLRYKSDSDKQQGPGVPSRFSGSKDASANAGIL | |
| | | LISGLQSEDEANYYCMIWHDSGWVFGGGTKLTVLGQPK | |

H = heavy chain variable region, K = kappa variable domain, and L = lambda variable domain

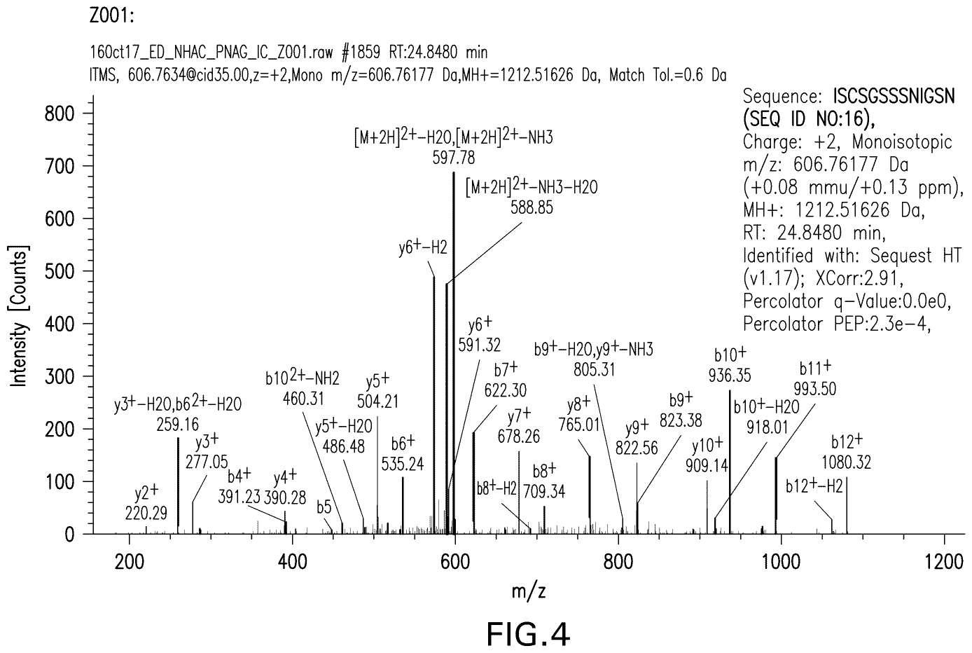

Reversed immune-capture followed by high resolution tandem mass spectrometry was used to determine the correct V_{H} domain/V_{L} domain pair for the PNAG-binding antibodies in the PNAG positive wells. Magnetic beads displaying PNAG were generated by reacting synthetic biotinylated PNAG (or dPNAG) 5-mers with commercial magnetic streptavidin beads. These PNAG beads were then used to selectively capture PNAG binding antibodies out of the antibody mixture present in the supernatant of the antibody secreting cells of each well following the scheme shown in FIG. 3 and described below. Unbound antibodies were removed by washing and bound antibody was eluted under low pH conditions. PNAG specific antibodies in the eluent were subjected to pepsin digestion to produce peptides and subsequent reduction of disulfide bridges between peptides. The resulting peptides of the PNAG binding antibody were then analyzed by high sensitivity reversed phase nano liquid chromatography tandem mass spectrometry (Ishihama, J. Chromatography A 1067: 73-83 (2005)). Tandem mass spectra of these peptides were matched against the RT-PCR derived V_{H} and V_{L} domain amino acid sequences of the well from which they were derived using the SEQUEST scoring algorithm (Tabb, J. Am. Soc. Mass Spectrometry 26: 1814-1819 (2015)). An example of the mass spectrometry spectra for peptide Z001-VL5 is shown in FIG. 4. We found that pepsin digestion produced superior results to digestion with trypsin and that the reduction of disulfide bonds after, as opposed to before, the digest further improved the sequence determination by mass spectrometry. The peptides obtained the PNAG binding antibody from PNAG positive well Z001 is shown in Table 5 and PNAG positive well C001 is shown in Table 6.

TABLE 5
Peptides identified for the PNAG binding antibody from PNAG
positive well Z001

| V_{L} Domain Peptides | Peptide Sequence | SEQ ID NO. |
|---|---|---|
| Z001-VL1 | AISGLQSDDEAD | 12 |
| Z001-VL2 | EGGLFGGGTKL | 13 |
| Z001-VL3 | LIHNNDQRPSGVPDRF | 14 |
| Z001-VL4 | LIHNNDQRPSGVPDRFSASKSGTSASL | 15 |
| Z001-VL5 | ISCSGSSSNIGSN | 16 |
| Z001-VL6 | TQPPSASGTPGQT | 17 |
| Z001-VL7 | TQPPSASGTPGQTVT | 18 |
| Z001-VL8 | YQHLPGTAPKL | 19 |
| V_{H} Domain Peptides | Peptide Sequence | SEQ ID NO. |
| Z001-VH1 | DIWGQGTM | 20 |
| Z001-VH2 | ESGGGLVQPGGSL | 21 |
| Z001-VH3 | WVRQAPGKGLE | 22 |
| Z001-VH4 | YADSVKGRF | 23 |
| Z001-VH5 | YLQMNSLRAGDTAV | 24 |
| Z001-VH6 | YYCAKDSCSHYDC | 25 |

TABLE 6
Peptides identified for the PNAG binding antibody from PNAG
positive well C001

| V_{L} Domain Peptides | Peptide Sequence | SEQ ID NO. |
|---|---|---|
| C001-VL1 | ISRLEPEDF | 26 |
| C001-VL2 | LAWYQQKPGQAPRL | 27 |
| C001-VL3 | YQQKPGQAPRL | 28 |
| C001-VL4 | SPGTLSLSPGERATLSCRASQSVYGSYLA | 29 |
| C001-VL5 | SLSPGERATL | 30 |
| C001-VL6 | YCQQYGSSPPYTFGPGT | 31 |
| C001-VL7 | KPGQAPRLLIYHASIRAT | 32 |
| C001-VL8 | GERATLSCRAS | 33 |
| C001-VL9 | YCQQYGSSPPYT | 34 |
| C001-VL10 | RFSGSGS | 35 |
| C001-VL11 | RATLSCRASQSVYGSYL | 36 |
| V_{H} Domain Peptides | Peptide Sequence | SEQ ID NO. |
| C001-VH1 | TISRDDSTNTAY | 37 |
| C001-VH2 | LVESGGGLVQPGGS | 38 |
| C001-VH3 | VGHIRNIADGSATAYSASVK | 39 |
| C001-VH4 | TNTAYLEMNSLRT | 40 |
| C001-VH5 | ESGGGLVQPGGSLRLSCAASGF | 4 |
| C001-VH6 | SSASTK | 42 |
| C001-VH7 | DTAVYYCSRQT | 43 |
| C001-VH8 | SCAASGFTVSGSHMHWVRQASGKGLE | 44 |
| | WVG | |
| C001-VH9 | SGKGLE | 45 |
| C001-VH10 | GSATAYSASVKGRFTI | 46 |
| C001-VH11 | RNIADGSATAYSASVKGRFTISRDDS | 47 |

By comparing the peptide amino acid sequences with the variable region amino acid sequences, the correct V_{H} domain/V_{L} domain pair for the PNG-binding antibody in PNAG positive well C001 comprises V_{H} C00-H1 and V_{L} C00-L1 shown in Table 3 and the correct V_{H} domain/V_{L} domain pair for the PNG-binding antibody in PNAG positive well Z001 comprises V_{H} Z00-H1 and V_{L} Z00-L1 shown in Table 4. The correct V_{H} domain/V_{L} domain pairs were similarly identified for the other PNAG positive wells using the scheme shown in FIG. 3. With one exception, the obtained tandem mass spectra exclusively identified the peptide sequences that corresponded to the variable regions of one unique V_{H} domain amino acid sequence and one unique V_{L} domain amino acid sequence, thereby identifying the correct domain pair amino acid sequences of the antibody. In the one exception (PNAG positive well C001), tandem mass spectra identified two possible V_{L} domain amino acid sequences. However, even in this case, the correct V_{L} domain amino acid sequence could be unambiguously determined as C001-L1 by ranking the sequence identifications based on their “summed peptide score” provided by SEQUEST. The “summed peptide score” of 4.79 for the binding antibody was 24-fold higher than the “summed peptide score” of the non-binding light chain sequence (Table 2).

To confirm that the correct heavy chain and light chain pair had been identified for each of the hits shown in Table 2, each of the possible heavy chain and light chain combinations were each separately transfected into Expi293F cells as described below and the antibodies obtained screened for binding to the antigen by ELISA against (a) a negative control comprising cell wall extract from a bacterium that doesn't express PNAG (S. epi strain PCIT200); (b) a positive control comprising cell wall extract from a bacterium that does express PNAG (as well as dPNAG) (S. epi PR62A); (c) synthetic PNAG (FIG. 1B); and (d) synthetic dPNAG (FIG. 1C).

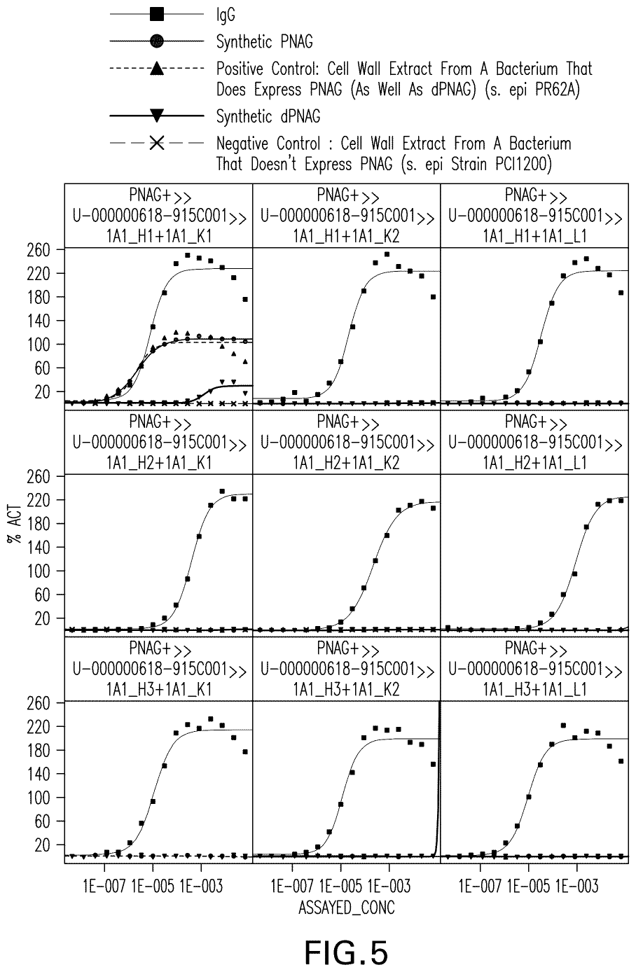

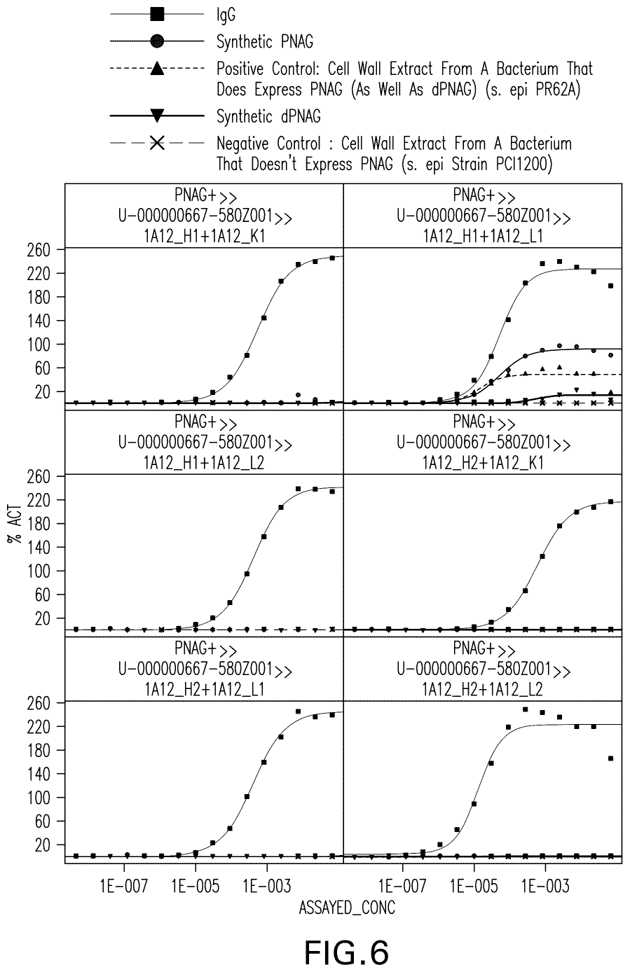

The ELISA results for the antibody heavy and light chains identified by NGS in PNAG positive wells C001 and Z001 are shown in FIG. 5 and FIG. 6, respectively. FIG. 5 confirms that the correct antibody pair for the PNG-binding antibody in PNAG positive well C001 comprises V_{H} domain C001-H1 and V_{L} domain C001-L1 shown in Table 3 since only the C001-H1 and C001-L1 combination had comparable binding to synthetic PNAG and the positive control. FIG. 6 confirms that the correct antibody pair for the PNG-binding antibody in PNAG positive well Z001 comprises V_{H} domain Z00-H1 and V_{L} domain Z00-L1 shown in Table 4 since only the Z001-H1 and Z001-L1 combination had comparable binding to synthetic PNAG and the positive control. The results confirmed that the method of the present invention had correctly identified the V_{H} and V_{L} domains making up the antibody in the positive wells that bound the antigen. In the method, peptide tandem mass spectra that mapped to conserved antibody amino acid sequences were not considered, since they did not allow distinguishing between different antibodies in the positive wells.

### Conclusion

The example demonstrates that the present invention is highly efficient for the identification of immunoglobulin heavy and light chain pairs of an antibody specific for a particular target a biological macromolecule and in each case was able to identify the correct heavy and light chain combination comprising the antibody species in B-cell positive wells that bind to a biological macromolecule from all other non-binding antibody species that may be in the well. While the antibodies used to exemplify the method had been made against a bacterial exopolysaccharide, the method may be used to identify antibodies from B-cell libraries that bind any a biological macromolecule or antigen. The present invention provides cost and time savings by omitting labor extensive combinatorial expression of all possible heavy and light chain pairs. Even higher cost and time savings can be realized by deliberately increasing the average number of plasma blasts per B cell screen well to more than one cell per well, for example, 1.4 cells per well or more, the upper limit currently being a limitation of the currently available software for analyzing the data and is expected to increase as more effective software for analyzing data becomes available.

### Materials and Methods

### Materials

Synthetic five-mer PNAG and dPNAG with linker and biotin moiety (FIG. 1B and FIG. 1C, respectively) were synthesized as fee for service by Corden Pharma (Corden Pharma, Boulder Colorado, USA). Pepsin was obtained from Promega (V1959 WI, USA).

Experimental Setup of the Revered Immunocapture High Resolution Tandem Mass Spectrometry (rIC/MS) Experiment

Preparation of PNAG magnetic beads and reversed immunocapture: 60 μL of streptavidin magnetic beads (Pierce; PN 8816) with a theoretical binding capacity of 2,100 pmol of substrate were washed five times with 150 μL of phosphate buffered saline (PBS; Merck in house) using a holder for 1.5 mL tubes with a strong magnet holder (Invitrogen). 3,175 pmol of biotinylated synthetic PNAG (or dPNAG) five-mer were dissolved in 135 μL of PBS and added to the washed magnetic beads. Biotinylated PNAG (dPNAG) five-mer was allowed to react with the Streptavidin magnetic beads for 20 minutes at room temperature in an incubator shaker (400 rpm). Excess biotinylated PNAG (dPNAG) five-mer was removed and the magnetic beads were washed five times with 200 μL of PBS. The washed PNAG (dPNAG): Streptavidin magnetic beads were taken up with 60 μL of PBS and vortexed to obtain a homogeneous mixture. 5 μL of the PNAG (dPNAG): Streptavidin magnetic bead mixture was pipetted into 200 μL of B cell supernatant obtained from the positive wells of the B-cell screen. Binding of the monoclonal antibody from the supernatant was allowed for one hour at room temperature in an incubator shaker, gently shaking at 400 rpm. Unbound antibody was removed after one hour of incubation and the magnetic beads were washed once for less than two minutes with 200 μL of PBS. The supernatant of this washing step was removed and the beads were immediately used for antibody digestion and identification.

Antibody digestion: 60 μL of digestion buffer (0.05N HCl) was added to the magnetic beads to release the PNAG bound antibody and the antibody release was performed for 10 minutes at RT while shaking at 400 rpm. The beads were retained from the supernatant fraction using a magnet and the supernatant fraction containing the released antibody was collected in a separate tube and placed for five minutes in a heating block set to 75° C. After cooling the sample down to room temperature, 1.5 μL (0.3 μg) aqueous pepsin solution (0.2 μg/μL) was added. The pepsin digestion was performed at 37° C. for a minimum of five hours or overnight (14 hours). Disulfide bonded peptides were reduced by adding 1.5 μL of 500 mM tris(2-carboxyethyl)phosphine (TCEP; Pierce, bond breaker PN 77720) and reacting for 15 minutes at 65° C. Digestion samples were then either stored at −80° C. or preferably analyzed immediately by 1D/LC-MS/MS.

Mass Spectrometry Data Acquisition: Mass spectrometry analysis was performed on an Orbitrap XL mass spectrometer (Thermo Scientific). The mass spectrometer was equipped with a nano electrospray source and directly coupled to a U300 RS nano HPLC (Dionex). The entire pepsin digest was loaded from the autosampler onto a reversed phase C18 trapping column (PepMap 100; 300 μm×5 mm; 5 μm particle sizes; 100 Å pore size). Sample trapping was performed for 8 minutes at a flow rate of 17 μL/minutes enabling salt removal. Peptides were then back eluted onto a C18 analytical column (Acclaim PepMap RSL 75 μm×15 cm, 2 μm particle size; 100 Å pore size) using a gradient increasing buffer b (100% acetonitrile and 0.1% formic acid) from 2% to 35% in 65 minutes. Buffer a consisted of 100% LC/MS grade water and 0.1% formic acid. Peptide elution was followed by a column cleanup step where buffer b was increased to 90% within 10 minutes and reconditioning of the analytical column to 2% buffer b within 20 minutes. Spectra acquisition was performed in data dependent manner with dynamic exclusion and charge state recognition enabled. Precursor ions were analyzed between m/z 400 and m/z 2000 at a resolution of 60.000 in the orbital ion trap. MS/MS scans of the top five parent m/z values were performed in the linear ion trap. Repeat analysis of m/z values within 15 ppm were excluded for 5 minutes. The mass accuracy and sensitivity of the mass spectrometer was tested before each measurement using the 6×5 peptide mixture from Promega (V7491). The instrument had to detect at least 3/6 peptides at 100 attomol or less and the mass accuracy for all detected peptides had to be better than 5 ppm prior to sample analysis.

Tandem Mass Spectra Analysis: Analysis of tandem mass spectra was performed using the software package Proteome Discoverer (Thermo Fisher, Version 2.2) using the SEQUEST HT search algorithm. A custom database was generated for each sample. The custom database contained the full length heavy and light chain sequences provided by NGS analysis as well as the bovine proteome. No enzyme specificity was selected and the pre-cursor mass had to match within 10 ppm. The monoisotopic signals of the tandem mass spectra had to match within 0.6 Da. Percolator was used to calculate false positive rates. The spectra analysis can be performed with any state of the art search engine for tandem mass spectra and is not confined to the software used here.

B-cell screening: Human memory B-cells were enriched from freshly isolated peripheral blood mononuclear cells (PBMC) as described previously (Xia et al., Oncotarget 8: 73654-73669 (2017)). Memory B-cells were seeded at an average of 1.4 B-cells/well in a total of 200 384-well plates, in the presence of 5,000 rad gamma irradiated HEK293 cells expressing human CD40 ligand at 10,000 cells/well and recombinant human IL-21 at 50 ng/mL and cultured for 14 days. B-cell culture supernatants were then collected and tested for binding to PNAG containing Staphylococcus epidermidis bacterial extract in primary screen.

Briefly, Staphylococcus epidermidis RP62A bacterial extract were immobilized at 1:1000 dilutions in PBS on 1536-well high-bind plates (Greiner Bio-One) at 4° C. overnight. Plates were blocked with 3% (vol/vol) nonfat milk in PBS/0.05% Tween-20 and then incubated with B-cell culture supernatants in five μL volume for 90 minutes at room temperature. Plates were washed afterwards with phosphate buffered saline (PBS)/0.05% Tween-20 and then Alkaline Phosphatase (AP)-conjugated goat anti-human IgG was added (Southern Biotech) at a dilution of 1:2000. Plates were incubated at room temperature for 60 min and washed. CDP-Star chemiluminescence reagent (PerkinElmer), an AP substrate, was added at 6 μL per well and incubate for 15 minutes. Plates were then read for luminescent signal on Envision (PerkinElmer). A sample with 10 fold or higher signal than the background control was scored as positive for that well.

The primary screen hits were cherry-picked and re-tested in a negative secondary screen against bacterial extract of a Staphylococcus epidermidis PNAG knock out strain as well as a positive secondary screen against synthetic PNAG and dPNAG five-mers using binding assays similar to the primary screen assay.

Expression of monoclonal antibodies for ELISA: Paired heavy chain and light chain plasmids were co-transfected into Expi293F cells (Thermo Fisher Scientific) for transient expression of antibodies following the manufacturer's protocol. Briefly, 0.5 μg of plasmid DNA (H:L chain DNA mixture at 1:2 ratio) was diluted in 25 μL of Opti-MEM I medium and mixed with 25 μL of ExpiFectamine 293 diluent containing 1.35 μL of ExpiFectamine 293 Reagent in Opti-MEM I medium. The DNA-ExpiFectamine 293 Reagent mixture was incubated at room temperature for 20-30 minutes and added to 96-well deep well plate containing 1.25×10⁶ cells of Expi293 cells in pre-warmed Expi293 expression medium per well. The plate was then covered with air porous tape (Thermos Fisher) and incubated at 37° C., 8% CO₂ with 93% humidity on MixMate shaker (Eppendorf) rotating at 1000 rpm for 20 hours, followed by the addition of 3.1 μL of ExpiFectamine 293 Transfection Enhancer 1 and 31.3 μL of ExpiFectamine 293 Transfection Enhancer 2 to each well, together with 0.25 mL of pre-warmed Expi293 Expression Medium containing 3× Pen/Strep. Supernatant fractions were harvested five days post-transfection by spinning down supernatants to sediment cell debris and subjected to testing in enzyme-linked immunosorbent assay (ELISA) binding assays as described above.

NGS of heavy and light chain sequences: Well-specific heavy chain PCR products were pooled and gel-purified. Kappa and Lambda PCR products were also pooled and gel-purified. The purified PCR products were pooled and sequenced on the MiSeq at GENEWIZ. The MiSeq output file was demultiplexed into chain- and well-specific FASTQ files using well-specific barcodes introduced in the final PCR step. The forward and reverse reads were assembled using Pear (Zhang et al., Bioinformatics (Oxford, England) 30: 614-620 (2014)) and the assembled reads processed by IgBlast (Ye et al., Nuc. Acids Res. 41: W34-40 (2013)) to identify the heavy and light chains. Non-antibody sequences and rare antibody sequences (less than 1% reads) were discarded. The remaining heavy and light chains were translated in-silico and compiled into databases (one sequence database per well). These databases were used to query the LC-MS/MS data from that well.

### Example 2

The example demonstrates operation of the present invention in monoclonal antibody hybridoma sample. In order to simulate a typical hybridoma screening sample, purified target monoclonal antibody specific for target antigen was combined with four unrelated monoclonal antibodies that do not bind the target antigen were combined at various concentrations. The total monoclonal antibody concentration was capped at 200 nM, which was based on an average of nine hybridoma supernatants. The purified target monoclonal antibody was tested at four concentrations: 0.2 nM (0.1% of total concentration), 2 nM (1% of total concentration), 20 nM (10% of total concentration) and 50 nM (25% of total concentration). A negative control sample, containing 1% of purified target monoclonal antibody, was prepared and added to beads that contained no biotinylated target antigen in order to ensure there was no non-specific binding between the monoclonal antibodies and the streptavidin coated magnetic beads. Two sets of samples were prepared in duplicate. One set was enzymatically digested with Pepsin and one set was enzymatically digested with Trypsin.

Preparation of hybridoma magnetic beads and reversed immunocapture: 150 μL of Dynabeads™ M-280 streptavidin magnetic beads (ThermoFisher Scientific; PN 60210) with a theoretical binding capacity of 650 to 900 pmoles of substrate per mg of beads were washed three times with 150 μL of phosphate buffered saline (PBS) using a holder for 96-well microtiter plate with a strong magnet holder (Invitrogen). 50 μL of 1 μM biotinylated target antigen were added to the washed magnetic beads along with 100 μL of 1×PBS. Biotinylated target antigen was allowed to react with the Streptavidin magnetic beads for 60 minutes at room temperature on a plate rotator. Excess biotinylated target antigen was removed and the magnetic beads were washed three times with 150 μL of 1×PBS. 100 μL of previously prepared samples were added directly to the washed beads. Binding of the monoclonal antibody from the supernatant was allowed for one hour at room temperature on a plate shaker. Unbound monoclonal antibody was removed after one hour of incubation and the magnetic beads were washed three times with 150 μL of PBS each time. Captured antibody was then eluted off the magnetic beads in acidic conditions.

Pepsin antibody digestion: 60 μL of digestion buffer (0.05N HCl) was added to the magnetic beads to release the captured monoclonal antibody and the antibody release was performed for 10 minutes at RT while shaking. The beads were retained from the supernatant fraction using a magnet and the supernatant fraction containing the released antibody was collected in a separate plate and placed for five minutes in a heating block set to 75° C. After cooling the sample down to room temperature, 5 μL (0.09 μg) aqueous pepsin (Promega; P/N V1959) solution (0.017 μg/μL) was added. The pepsin digestion was performed at 37° C. overnight (16 hours). The plate was then placed in a heating block set to 95° C. for 10 minutes to deactivate the pepsin. Disulfide bonded peptides were reduced by adding 3 μL of 250 mM tris(2-carboxyethyl)phosphine (TCEP; Pierce, bond breaker PN 77720) and reacting for 15 minutes at 65° C. Digestion samples were then either stored at −80° C. or preferably analyzed immediately by 1D/LC-MS/MS.

Trypsin antibody digestion: 50 μL of elution buffer (0.1% Trifluoracetic acid) was added to the magnetic beads to release the captured monoclonal antibody and the antibody release was performed for 10 minutes at RT while shaking. The beads were retained from the supernatant fraction using a magnet and the supernatant fraction containing the released antibody was collected in a separate plate. 2 μL of Tris-HCl pH 9 buffered solution was added to neutralize the samples. 40 μL of a 0.2% RAPIGEST (Waters Corp., P/N 186008090) prepared in a 50 mM Ammonium Bicarbonate (Millipore Sigma, P/N 09830) was added to each well. Disulfide bonded peptides were reduced by adding 2 μL of 250 mM Dithiothreitol (ThermoScientific Peirce, P/N A39255) and reacting for 45 minutes at 65′C. After cooling the sample down to room temperature, 3 μL of an alkylating reagent, iodoacetamide (A3221-1VL Sigma Aldrich) was added to block the re-formation of cystine bonds and allowed to react for 30 minutes at room temperature in the dark. 5 μL (0.09 μg) aqueous mass-spectrometer grade Trypsin gold (Promega; P/N V5280) solution (0.017 μg/μL) was added. The trypsin digestion was performed at 37° C. overnight (16 hours). After digestion, to remove the RAPIGEST 1 μL of Triflouroacetic acid was added to the samples and the plate was incubated at 37° C. for 45 minutes. After incubation, the plate was centrifuged at 10,000×RPM for 15 minutes at room temperature to pellet the RAPIGEST and the supernatant containing the digested monoclonal antibody was removed. Digestion samples were then either stored at −80° C. or preferably analyzed immediately by 1D/LC-MS/MS.

Mass Spectrometry Data Acquisition: Mass spectrometry analysis was performed on a Thermo Orbitrap Fusion Lumos Tribrid mass spectrometer (Thermo Scientific). The mass spectrometer was equipped with a nano electrospray source and directly coupled to a NanoAcquity HPLC (Waters). 1 μL was loaded from the autosampler onto a reversed phase C18 trapping column Waters nanoAQUITY UPLC 2G-V/Mtrap 5 μm Symmetry C18 180 μm×20 mm. Sample trapping was performed for five minutes at a flow rate of 10 μL/minute enabling salt removal. Peptides were then back eluted onto a C18 analytical column (PicoChip REPROSIL-Pur-C18-AQ 3 μm 120A; 75 μm×105 mm) at a flow rate of 500 nL/min using a gradient increasing buffer B (100% acetonitrile and 0.1% formic acid) from 0.5% to 35% in 22 minutes. Buffer A consisted of 100% LC/MS grade water and 0.1% formic acid. Peptide elution was followed by a column cleanup step where buffer B was increased to 80% at 23 minutes and held for 4 minutes. Reconditioning of the analytical column to 0.5% buffer B occurred from 27.1 minutes to 60 minutes. Spectra acquisition was performed in data dependent manner with dynamic exclusion and charge state recognition enabled. Precursor ions were analyzed between m/z 375 and m/z 1800 at a resolution of 240,000 in the orbital ion trap. MS/MS scans of the parent m/z values were performed in the linear ion trap. Repeat analysis of m/z values within +/−5 ppm were excluded for 1 minute.

Tandem Mass Spectra Analysis: Analysis of tandem mass spectra was performed using the software package Mascot Distiller (Matrix Science, Version 2.7.1.0 (64-bit)). A custom database was generated, containing the variable region of the heavy chain and the variable region of the kappa light chain for the monoclonal antibodies used in the experiment, as well as the human IgG1 constant region of the heavy chain and the constant region of the Kappa light chain for both mouse and human. Either trypsin or pepsin enzyme specificity was selected based on the data file and the pre-cursor mass had to match within 20 ppm. The monoisotopic signals of the tandem mass spectra had to match within 0.3 Da. For trypsin digested samples, carbamidomethyl was added as a fixed modification and acetyl (Protein N-Term) and Oxidation (M) were added as variable modifications. For Pepsin digested samples, acetyl (Protein N-Term) and Oxidation (M) were added as variable modifications. The spectra analysis can be performed with any state-of-the-art search engine for tandem mass spectra and is not confined to the software used here.

### Results

This example demonstrates that the present invention can determine the sequence of the heavy and light chains of a monoclonal antibody specifically binding a target biological macromolecule from among a plurality of antibodies that do not bind the target biological macromolecule in a hybridoma sample plated at a cell density greater than one cell per well. In every experimental sample, the correct heavy and light chain pair corresponding to the purified target monoclonal antibody was identified. For the replicates digested using trypsin (Table 7), the sequence coverage of the variable region of the heavy chain ranged from 33% to 44% with between three to seven unique peptides being identified. For the kappa light chain, the sequence coverage was 8% across the board with the same one unique peptide being identified. For the replicates digested using pepsin (Table 8), the sequence coverage of the variable region of the heavy chain ranged from 21% to 88% with between one to 10 unique peptides being identified. For the kappa light chain, the sequence coverage of the variable region ranged from 11% to 30% with either one, two, or three unique peptides being identified. The summarized results and unique peptides are listed in the Tables 7 and 8. The negative control showed no non-specific binding of the antibodies to the magnetic beads. Being able to identify monoclonal antibodies at sub-cloning/sequencing stage using reverse capture MS plus next generation sequencing (NGS) facilitates the sub-cloning process (resource saving) and may lead to the identification of more unique monoclonal antibodies.

TABLE 7
Trypsin Results

| Total | | | | Heavy Chain | | Light Chain | |
|---|---|---|---|---|---|---|---|
| sample | Conc of | | | Ave | Ave # | Ave | Ave # |
| concen- | Positive | | | % seq | unique | % seq | unique |
| tration | Hybridoma | | n | Coverage | peptides | Coverage | peptides |
| 200 nM - | 2 | nM | 4 | Not | | Not | |
| no antigen | | | | identified | | identified | |
| control | | | | | | | |
| 200 nM | 0.2 | nM | 4 | 36 | 3.8 | 8 | 1 |
| 200 nM | 2 | nM | 4 | 44 | 6 | 8 | 1 |
| 200 nM | 20 | nM | 4 | 42.8 | 5.8 | 8 | 1 |
| 200 nM | 50 | nM | 4 | 42.5 | 6.5 | 8 | 1 |

TABLE 8
Pepsin Results

| Total | | | | Heavy Chain | | Light Chain | |
|---|---|---|---|---|---|---|---|
| sample | Conc of | | | Ave | Ave # | Ave | Ave # |
| concen- | Positive | | | % seq | unique | % seq | unique |
| tration | Hybridoma | | n | Coverage | peptides | Coverage | peptides |
| 200 nM - | 2 | nM | 4 | Not | | Not | |
| no antigen | | | | identified | | identified | |
| Control | | | | | | | |
| 200 nM | 0.2 | nM | 4 | 15.5 | 1.3 | 39 | 3.8 |
| 200 nM | 2 | nM | 4 | 23.3 | 2 | 75 | 8.3 |
| 200 nM | 20 | nM | 4 | 26.3 | 2.3 | 78 | 8.8 |
| 200 nM | 50 | nM | 4 | 29.5 | 2.8 | 83 | 8.3 |

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

What is claimed:

1. A method for identifying an antibody variable heavy (V_{H}) chain and variable light (V_{L}) chain pair specific for an antigen of interest from a library of antibody producing memory B-cells, comprising the steps of:
(a) providing a library of antibody producing memory B-cells that produce human or animal antibodies dispensed into the wells of one or more multiwell plates at a density of 1.4 cells/well wherein the antibody producing memory B-cells are grown in a culture medium under conditions sufficient for the antibody producing memory B-cells to express antibodies into the culture medium;
(b) obtaining a first sample of culture medium from each of the wells and reacting the culture medium with the antigen of interest to identify those wells that contain antibodies that bind the antigen of interest;
(c) producing the nucleotide and amino acid sequences encoding the VH and VL chains of the antibodies from those wells that contain antibodies that bind the antigen of interest by
(i) lysing the antibody producing cells to release mRNA from the antibody producing cells;
(ii) using reverse transcription polymerase chain reaction (RT-PCR) amplification and a nucleic acid primer pairs specific for V_{H} and V_{L} chains of antibodies to make cDNA copies of the mRNA encoding the V_{H} and V_{L} chains of antibodies; and
(iii) sequencing the cDNA copies to provide polynucleotide sequences encoding the V_{H} and V_{L} chains and translating the polynucleotide sequences encoding the V_{H} and V_{L} chains to provide predicted amino acid sequences of the V_{H} chains and V_{L} chains;
(d) determining a theoretical tandem mass spectrometry spectra for the peptides that would be predicted from a pepsin digest of each of the predicted VH and VL chain amino acid sequences;
(e) obtaining a second sample of culture medium from those wells that contain antibodies that bind the antigen of interest and contain more than one species of V_{H} chain or V_{L} chain from step (c), reacting the culture medium with the antigen of interest immobilized to a solid support to bind and separate the antibodies that bind the antigen of interest from antibodies that do not bind the antigen of interest;
(f) releasing the antibodies bound to the antigen of interest on the solid support, digesting the antibodies with pepsin to produce peptide fragments, and subsequently reducing disulfide bonded peptide fragments thereof with tris(2-carboxyethyl)phosphine (TCEP) to produce reduced peptide fragments; and
(g) identifying the amino acid sequences of the reduced peptide fragments from step (f) by tandem mass spectrometry and comparing the tandem mass spectra for each of the reduced peptide fragments to the theoretical spectra for each of the peptides from step (d) to identify the amino acid sequences for the V_{H} chain and V_{L} chain pair specific for the antigen of interest.

2. The method of claim 1, wherein the primer pair comprises an upstream PCR primer and a downstream PCR primer wherein the melting temperature difference (ΔTm) between the upstream primer and the downstream primer is 3° C. or less.

3. The method of claim 1, wherein each well is assigned a specific nucleotide sequence identifier and one or both of the upstream and downstream PCR primers for each well comprises the specific nucleotide sequence identifier assigned to the well.

4. A method for determining the amino acid sequences of an antibody variable heavy (V_{H}) chain and variable light (V_{L}) chain pair specific for an antigen of interest from other antibodies produced by a mixture of antibody producing cells, comprising:
(a) providing a memory B-cell culture of a mixture of antibody producing cells that produce human or animal antibodies derived from two to 10 starting antibody producing memory B-cells, wherein each starting antibody producing memory B-cell expresses an antibody and wherein one of the two or more starting antibody producing memory B-cells expresses the antibody specific for the antigen of interest and the other two or more starting antibody producing cells express antibodies that do not bind the antigen of interest;
(b) producing the nucleotide and amino acid sequences encoding the VH and VL chains of the antibodies from the memory B-cell culture by
(i) lysing the antibody producing B cells to release mRNA from the antibody producing memory B-cells;
(ii) using reverse transcription polymerase chain reaction (RT-PCR) amplification and nucleic acid primer pairs specific for V_{H} and V_{L} chains of antibodies to make cDNA copies of the mRNA molecules encoding the V_{H} and V_{L} chains of antibodies; and
(iii) sequencing the cDNA copies to provide polynucleotide sequences encoding the V_{H} and V_{L} chains and translating the polynucleotide sequences encoding the V_{H} and V_{L} chains to provide predicted amino acid sequences of the V_{H} chains and V_{L} chains;
(c) determining a theoretical tandem mass spectrometry spectra for each of the peptides that would be predicted from a pepsin digest of each of the predicted amino acid sequences of the VH and VL chains;
(d) obtaining the antibodies that bind the antigen of interest from the B cell culture of step (a) by contacting the B cell culture with the antigen of interest immobilized to a solid support to separate the antibodies that bind the antigen of interest from those antibodies that do not bind the antigen of interest;
(e) digesting the antibodies that bind the antigen of interest with pepsin to produce peptide fragments and subsequently reducing disulfide bonded peptide fragments thereof with tris(2-carboxyethyl)phosphine (TCEP) to produce reduced peptide fragments; and
(f) identifying the amino acid sequences of the reduced peptide fragments from step (e) by tandem mass spectrometry and comparing the tandem mass spectra for each of the reduced peptide fragments to the theoretical tandem mass spectra from step (c) for each of the V_{H} and V_{L} chains from step (e) to identify the amino acid sequences for the V_{H} chain and V_{L} chain pair specific for the antigen of interest.

5. The method of claim 4, wherein the RT-PCR is performed with a primer pair comprising an upstream PCR primer and a downstream PCR primer wherein the melting temperature difference (ΔTm) between the upstream primer and the downstream primer is 3° C. or less.

6. The method of claim 4, wherein the B cell culture is assigned a specific nucleotide sequence identifier and one or both of the upstream and downstream PCR primers for the B cell culture comprises the specific nucleotide sequence identifier assigned to the well.

7. A method for obtaining the amino acid sequences of a human or animal antibody specific for an antigen of interest from the amino acid sequences of other antibodies produced by a mixture of antibody producing memory B-cells, comprising:
(a) providing a cell culture of a mixture of human or animal antibody producing memory B-cells derived from two to 10 starting antibody producing memory B cells, wherein each starting antibody producing memory B cell expresses an antibody and wherein one of the starting antibody producing memory B cells expresses the antibody specific for the antigen of interest and the other starting antibody producing memory B cells express antibodies that do not bind the antigen of interest;
(b) producing the nucleotide and amino acid sequences encoding the antibodies from the antibody producing memory B-cell culture by
(i) lysing the antibody producing memory B-cells to release mRNA from the antibody producing memory B-cells;
(ii) using reverse transcription polymerase chain reaction (RT-PCR) amplification and nucleic acid primer pairs specific for V_{H} and V_{L} chains of antibodies to make cDNA copies of the mRNA encoding the V_{H} and V_{L} chains of antibodies; and
(iii) sequencing the cDNA copies to provide polynucleotide sequences encoding the V_{H} and V_{L} chains and translating the polynucleotide sequences encoding the V_{H} and V_{L} chains to provide predicted amino acid sequences of the V_{H} chains and V_{L} chains;
(c) determining a theoretical tandem mass spectrometry spectra for each of the peptides that would be predicted from a pepsin digest of each of the predicted amino acid sequences of the VH and VL chains;
(d) obtaining the antibodies that bind the antigen of interest from the antibody producing memory B-cell culture by reacting the antibody producing memory B-cell culture with the antigen of interest immobilized to a solid support to separate the antibodies that bind the antigen of interest from the antibodies that do not bind the antigen of interest;
(e) digesting the antibodies that bind the antigen of interest with pepsin to produce peptide fragments and subsequently reducing disulfide bonded peptide fragments thereof with tris(2-carboxyethyl)phosphine (TCEP) to produce reduced peptide fragments; and
(f) identifying the amino acid sequences of the reduced peptide fragments from step (e) by tandem mass spectrometry and comparing the tandem mass spectra for each of the reduced peptide fragments to the theoretical tandem mass spectra from step (c) for each antibody from step (e) to determine the amino acid sequences of the antibody specific for the antigen from the mixture of antibodies.

8. The method of claim 7, wherein the RT-PCR is performed with a primer pair comprising an upstream PCR primer and a downstream PCR primer wherein the melting temperature difference (ΔTm) between the upstream primer and the downstream primer is 3° C. or less.


SEQUENCE LISTING (47 sequences)

SEQ ID NO: 1 - protein, 123 aa, synthetic construct
  REGION 1..123: note = Polypeptide
EVQLVESGGGLVQPGGSLRLSCAASGFTVSGSHMHWVRQASGKGLEWVGHIRNIADGSATAYSASVKGRFTISRDDSTNTAYLEMNSLRTEDTAVYYCSRQTLSCHDYWGQGALVTVSSASTK

SEQ ID NO: 2 - protein, 136 aa, synthetic construct
  REGION 1..136: note = Polypeptide
QVQLQESGPRRVKPSETLSLTCSVSGGSVRSGNYYWNWIRQPPGKGLEWIGYIFDSGTTDYNPSLKSRVTMSVDTSKNQFSLQLRSVTTADTAVYYCVREEGKLGDCSDGSCRRKYFWFDPWGQGTLVTVSSASTK

SEQ ID NO: 3 - protein, 131 aa, synthetic construct
  REGION 1..131: note = Polypeptide
EVQLVQSGGEVKKPGESLRISCKGSGYSFTNYWISWVRQMPGKGLEWMGKIDPSDSYTKYSPSFQGHVTISADKSISTAYLQWSSLKASDTAMYYCARHGSRDVVEVPAAFNWFDPWGQGTLVTVSSASTK

SEQ ID NO: 4 - protein, 110 aa, synthetic construct
  REGION 1..110: note = Polypeptide
EIVLTQSPGTLSLSPGERATLSCRASQSVYGSYLAWYQQKPGQAPRLLIYHASIRATGIPDRFSGSGSGTDFTLTISRLEPEDFAVYYCQQYGSSPPYTFGPGTKVDIKR

SEQ ID NO: 5 - protein, 113 aa, synthetic construct
  REGION 1..113: note = Polypeptide
DIQMTQSPSSLSASLGDRVIITCRASQNINRNLNWYQQKPGKAPQLLIYAASTLQSGVPSRFSGSGSGTDFSLTISSLQPGDFATYYCQQSSSTPRTFGQGTKVEIKRTIGKV

SEQ ID NO: 6 - protein, 114 aa, synthetic construct
  REGION 1..114: note = Polypeptide
QSALTQPRAVSGSPGQSVTISCTGTSSNVGRYNYVSWYQQRPGKAPTLMIYDVSERPSGVPDRFSGSKSGNTASLTISGLQADDEADYYCCSYAGSYTLLFGGGTKLTVLGQPK

SEQ ID NO: 7 - protein, 129 aa, synthetic construct
  REGION 1..129: note = Polypeptide
EVQLVESGGGLVQPGGSLRLSCAASGFSFTYAMSWVRQAPGKGLEWVSAISETGDYIYYADSVKGRFTISRDYSKSTLYLQMNSLRAGDTAVYYCAKDSCSHYDCYTRGFGFDIWGQGTMVTVSSASTK

SEQ ID NO: 8 - protein, 126 aa, synthetic construct
  REGION 1..126: note = Polypeptide
QVQLVQSGAEVRKPGSSMKVSCKASGGTFSTYGFHWVREAPGQGLEWVGGIMVVFDTTYYAQKFQGRVTITADEFTSTVYMEMSSLTSEDTAVYYCARDPYYDYEQGQWDVWGKGTTVTVSSASTK

SEQ ID NO: 9 - protein, 108 aa, synthetic construct
  REGION 1..108: note = Polypeptide
DIQMTQSPSSVSASVGDRVTITCRASQGISSWLAWYQQKPGKAPKLLIYAASSLQSGVPSRFSGSGSGTDFTLTISSLQPEDFATYYCQQANSFPVTFGQGTRLEIKR

SEQ ID NO: 10 - protein, 114 aa, synthetic construct
  REGION 1..114: note = Polypeptide
QSVLTQPPSASGTPGQTVTISCSGSSSNIGSNTVNWYQHLPGTAPKLLIHNNDQRPSGVPDRFSASKSGTSASLAISGLQSDDEADYYCAVWDDSLEGGLFGGGTKLTVLGQPK

SEQ ID NO: 11 - protein, 119 aa, synthetic construct
  REGION 1..119: note = Polypeptide
QAVLTQPSSLSASPGASASLTCTLHSGINVATYRIFWYQQKPGSPPHYLLRYKSDSDKQQGPGVPSRFSGSKDASANAGILLISGLQSEDEANYYCMIWHDSGWVFGGGTKLTVLGQPK

SEQ ID NO: 12 - protein, 12 aa, synthetic construct
  REGION 1..12: note = Polypeptide
AISGLQSDDEAD

SEQ ID NO: 13 - protein, 11 aa, synthetic construct
  REGION 1..11: note = Polypeptide
EGGLFGGGTKL

SEQ ID NO: 14 - protein, 16 aa, synthetic construct
  REGION 1..16: note = Polypeptide
LIHNNDQRPSGVPDRF

SEQ ID NO: 15 - protein, 27 aa, synthetic construct
  REGION 1..27: note = Polypeptide
LIHNNDQRPSGVPDRFSASKSGTSASL

SEQ ID NO: 16 - protein, 13 aa, synthetic construct
  REGION 1..13: note = Polypeptide
ISCSGSSSNIGSN

SEQ ID NO: 17 - protein, 13 aa, synthetic construct
  REGION 1..13: note = Polypeptide
TQPPSASGTPGQT

SEQ ID NO: 18 - protein, 15 aa, synthetic construct
  REGION 1..15: note = Polypeptide
TQPPSASGTPGQTVT

SEQ ID NO: 19 - protein, 11 aa, synthetic construct
  REGION 1..11: note = Polypeptide
YQHLPGTAPKL

SEQ ID NO: 20 - protein, 8 aa, synthetic construct
  REGION 1..8: note = Polypeptide
DIWGQGTM

SEQ ID NO: 21 - protein, 13 aa, synthetic construct
  REGION 1..13: note = Polypeptide
ESGGGLVQPGGSL

SEQ ID NO: 22 - protein, 11 aa, synthetic construct
  REGION 1..11: note = Polypeptide
WVRQAPGKGLE

SEQ ID NO: 23 - protein, 9 aa, synthetic construct
  REGION 1..9: note = Polypeptide
YADSVKGRF

SEQ ID NO: 24 - protein, 14 aa, synthetic construct
  REGION 1..14: note = Polypeptide
YLQMNSLRAGDTAV

SEQ ID NO: 25 - protein, 13 aa, synthetic construct
  REGION 1..13: note = Polypeptide
YYCAKDSCSHYDC

SEQ ID NO: 26 - protein, 9 aa, synthetic construct
  REGION 1..9: note = Polypeptide
ISRLEPEDF

SEQ ID NO: 27 - protein, 14 aa, synthetic construct
  REGION 1..14: note = Polypeptide
LAWYQQKPGQAPRL

SEQ ID NO: 28 - protein, 11 aa, synthetic construct
  REGION 1..11: note = Polypeptide
YQQKPGQAPRL

SEQ ID NO: 29 - protein, 29 aa, synthetic construct
  REGION 1..29: note = Polypeptide
SPGTLSLSPGERATLSCRASQSVYGSYLA

SEQ ID NO: 30 - protein, 10 aa, synthetic construct
  REGION 1..10: note = Polypeptide
SLSPGERATL

SEQ ID NO: 31 - protein, 17 aa, synthetic construct
  REGION 1..17: note = Polypeptide
YCQQYGSSPPYTFGPGT

SEQ ID NO: 32 - protein, 18 aa, synthetic construct
  REGION 1..18: note = Polypeptide
KPGQAPRLLIYHASIRAT

SEQ ID NO: 33 - protein, 11 aa, synthetic construct
  REGION 1..11: note = Polypeptide
GERATLSCRAS

SEQ ID NO: 34 - protein, 12 aa, synthetic construct [sequence omitted]

SEQ ID NO: 35 - protein, 7 aa, synthetic construct
  REGION 1..7: note = Polypeptide
RFSGSGS

SEQ ID NO: 36 - protein, 17 aa, synthetic construct
  REGION 1..17: note = Polypeptide
RATLSCRASQSVYGSYL

SEQ ID NO: 37 - protein, 12 aa, synthetic construct
  REGION 1..12: note = Polypeptide
TISRDDSTNTAY

SEQ ID NO: 38 - protein, 14 aa, synthetic construct
  REGION 1..14: note = Polypeptide
LVESGGGLVQPGGS

SEQ ID NO: 39 - protein, 20 aa, synthetic construct
  REGION 1..20: note = Polypeptide
VGHIRNIADGSATAYSASVK

SEQ ID NO: 40 - protein, 13 aa, synthetic construct
  REGION 1..13: note = Polypeptide
TNTAYLEMNSLRT

SEQ ID NO: 41 - protein, 22 aa, synthetic construct
  REGION 1..22: note = Polypeptide
ESGGGLVQPGGSLRLSCAASGF

SEQ ID NO: 42 - protein, 6 aa, synthetic construct
  REGION 1..6: note = Polypeptide
SSASTK

SEQ ID NO: 43 - protein, 11 aa, synthetic construct
  REGION 1..11: note = Polypeptide
DTAVYYCSRQT

SEQ ID NO: 44 - protein, 29 aa, synthetic construct
  REGION 1..29: note = Polypeptide
SCAASGFTVSGSHMHWVRQASGKGLEWVG

SEQ ID NO: 45 - protein, 6 aa, synthetic construct
  REGION 1..6: note = Polypeptide
SGKGLE

SEQ ID NO: 46 - protein, 16 aa, synthetic construct
  REGION 1..16: note = Polypeptide
GSATAYSASVKGRFTI

SEQ ID NO: 47 - protein, 26 aa, synthetic construct
  REGION 1..26: note = Polypeptide
RNIADGSATAYSASVKGRFTISRDDS